(19) United States
(12) Patent Application Publication
(10) Pub. No.: US 2010/0317996 A1
(43) Pub. Date: Dec. 16, 2010

(54) ULTRASOUND-NAVIGABLE BARBED BIOPSY DEVICE AND METHOD

(76) Inventor: Travis E. Dillon, Winston-Salem, NC (US)

Correspondence Address: BRINKS HOFER GILSON & LIONE/CHICAGO/COOK, PO BOX 10395, CHICAGO, IL, 60610, US

(21) Appl. No.: 12/482,076

(22) Filed: Jun. 10, 2009

(51) Int. Cl. A61B 10/02

(52) U.S. Cl. 600/567

(57) ABSTRACT

A tissue sampling system includes an elongate flexible needle sheath with a lumen disposed longitudinally therethrough. A needle may be slidably disposed through the needle sheath and include an elongate flexible shaft body configured to pass through a working channel of an endoscope, with an outer cannula disposed at a distal end of the shaft body and housing a longitudinal needle lumen though its length. A sampling member including an elongate flexible stylet body and a barbed portion extending distally from the stylet body may be disposed slidably through the needle lumen. It may include a body with a distal end and barbs forming a barbed region where the barbs are angled away from the distal end. The barbed region may be configured to be slidably extended out the distal end of the needle lumen, then retracted, and may include an echogenic surface along with the barbs and barb-adjacent surfaces.

### TECHNICAL FIELD

The invention relates generally to medical devices useful for obtaining a biopsy tissue sample. More particularly, the invention pertains to a barbed tissue-sampling device and needle cannula system configured for deployment within a patient body.

### BACKGROUND

Biopsy of tissue from a patient can be an important means of obtaining diagnosis of a disease condition. Minimally invasive surgical methods of obtaining biopsied tissue samples have been developed that may lessen the discomfort a patient suffers during a biopsy procedure, and that often reduces the risk of infection by minimizing or eliminating punctures or incisions in the patient's external integument. Endoscopic methods such as those using gastro-esophageal endoscopy may use fine-needle aspiration (FNA) to obtain cell and/or tissue samples (where the tissue sample is typically larger maintains more integrity between the cells therein). As used herein, “tissue sample” includes both cell samples and tissue samples. When obtaining a tissue sample by FNA, it is often necessary to pass the needle several times into/through the target tissue site (e.g., tumor) in order to get a sufficient sample for diagnostic purposes. This repetitive process may lengthen the time of the procedure, introducing inefficiencies for the patient and attending medical personnel, and it may increase the discomfort suffered by a patient during the procedure.

It is desirable to maximize efficiency of biopsy procedures by providing a device to obtain a desirable tissue sample with as few operative actions and as little time as possible.

### BRIEF SUMMARY

A tissue sampling system may include an elongate flexible needle sheath with a lumen disposed longitudinally therethrough. A needle may be slidably disposed through the needle sheath and include an elongate flexible shaft body configured to pass through a working channel of an endoscope, with an outer cannula disposed at a distal end of the shaft body housing a longitudinal shaft lumen though its length. A sampling member including an elongate flexible stylet body and a barbed portion extending distally from the stylet body may be disposed slidably through the needle lumen. It may include a body with a distal end and barbs forming a barbed region where the barbs are angled away from the distal end. The barbed region may be configured to be slidably extended out the distal end of the needle lumen, then retracted, and may include an echogenic surface.

### BRIEF DESCRIPTION OF THE DRAWINGS

FIG. 1A shows a prior art barbed broach;

FIG. 1 shows a tissue sampling device;

FIG. 2 shows a barbed tissue sampling device embodiment;

FIGS. 3, 3A, and 3B show another barbed tissue sampling device embodiment, where FIGS. 3A-3B show transverse section views of the tissue sampling device of FIG. 3;

FIG. 4 shows a method of obtaining a tissue sample;

FIG. 5 shows a further method of obtaining a tissue sample;

FIG. 6 shows a side view of a tissue sampling device and needle; and

FIG. 6A shows an end view of the tissue sampling device and needle of FIG. 6.

### DETAILED DESCRIPTION

As used herein, “proximal” refers to an end or direction associated with a physician or other treating personnel during a device operation, and “distal” refers to the opposite end (“patient end/treating end”). The term “tissue sampling device” is used generally to refer to biopsy devices. The drawing figures referred to herein are provided for illustrative purpose only. They should not be construed as limiting the scope of the invention defined by the claims, including that they may not necessarily be drawn to scale.

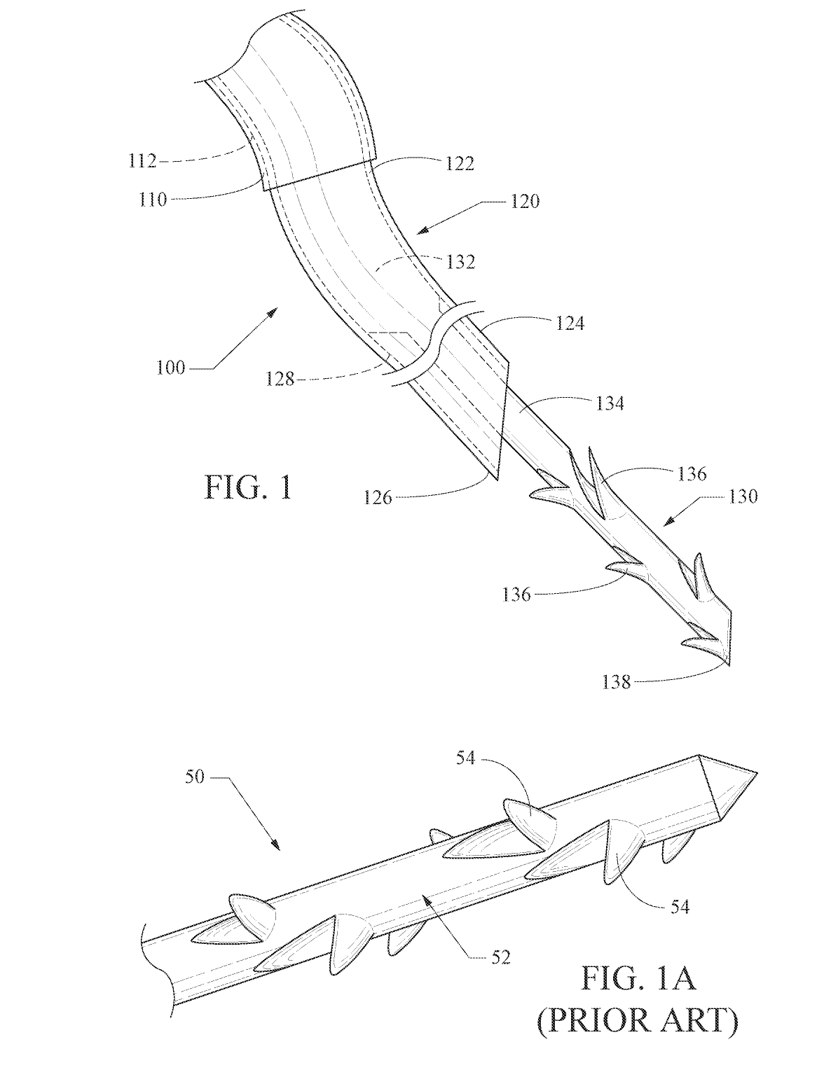

A device known as a broach or barbed broach is known from the dental art, where it is used to remove pulp tissue for disposal during root canal procedures. In its simplest form, a prior art barbed broach, such as is illustrated in FIG. 1A, may be a generally cylindrical rigid metal rod 50 that has had barbs 54 formed its surface 52 by divoting and slightly levering out portions of the rod body. This prior art device has been known for decades, is wholly unrelated to the endoscopy art, and is not used for tissue sampling. However, the present inventor discovered surprising advantages in providing a barbed device with enhanced echogenicity used together with a sheath to sample tissue endoscopically, and particularly to sample tissues in gastrointestinal endoscopic procedures with advantages of simplicity and economic feasibility as contracted with mechanically more complex biopsy forceps and the like.

A tissue sampling device embodiment 100 is described with reference to FIG. 1. The tissue sampling device 100 includes a needle sheath 110, which is a flexible elongate tubular structure including a sheath lumen 112. An elongate member embodied as a needle member 120 may be slidably disposed through the sheath lumen 112. The needle member 120 preferably includes an elongate flexible needle shaft body 122. A needle cannula 124, illustrated here with a beveled sharp penetrating distal end 126, extends distally from the flexible needle shaft body 122. The needle member 120 includes a needle lumen 128. A sampling member 130 is slidably disposed through the needle lumen 128. The sampling member includes an elongate flexible stylet body 132. A barbed region 134 extends distally from the stylet body 132. Barbs 136 on the barbed region 134 preferably angle away from the distal member tip 138, which is shown as being beveled.

The tissue sampling device 100 preferably is configured for passage through the working channel of an endoscope such as, for example, the type used in gastrointestinal and/or biliary tract procedures. Accordingly, the dimensions and flexibility of the device may be adapted for particular endoscope models. In other embodiments that will readily be appreciated in view of the present disclosure and knowledge of those having skill in the art, the device may be configured for introduction directly into/through a natural body orifice (e.g., urethra).

FIG. 2 shows another embodiment of a barbed tissue sampling device 200. It includes a generally conical distal tip 202. The device body 204 is generally cylindrical. It includes a coarse barb 208. The coarse barb 208 is configured such that a radius (line R) from the longitudinal body axis (dashed line 210) to an outermost barb tip 208a is at least the same as or greater than a distance equal to the device body outer diameter (the length of line OD). The barbed tissue sampling device 200 also includes a round-tipped barb 212, while other barbs may include one or more sharpened points and rounded or cutting edges.

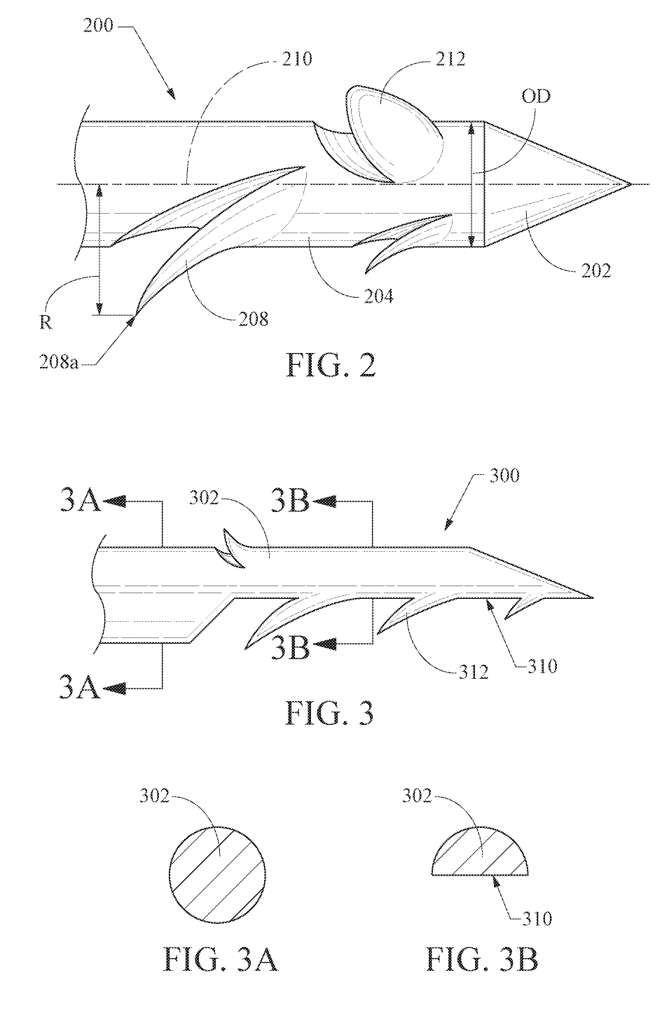

FIGS. 3-3B show another embodiment of a barbed tissue sampling device 300. The tissue sampling device 300 includes a non-cylindrical geometry. As shown in the transverse section of FIG. 3A, taken along line 3A-3A of FIG. 3, a portion of the device body 302 includes a circular cross-section. However, as shown in FIG. 3B, which is a transverse section view taken along line 3B-3B of FIG. 3, a distal portion of the device body 302 has a non-cylindrical geometry, instead being formed as a semi-cylinder (viewed as a semi-circle in the section view of FIG. 3B). In other embodiments, the non-cylindrical geometry may be embodied as a body having a triangular or other polygonal or other geometric cross-section, or combination of different cross-sectional geometries. The tissue sampling device 300 includes a generally flat planar face 310. A plurality of barbs 312 extends from the face 310.

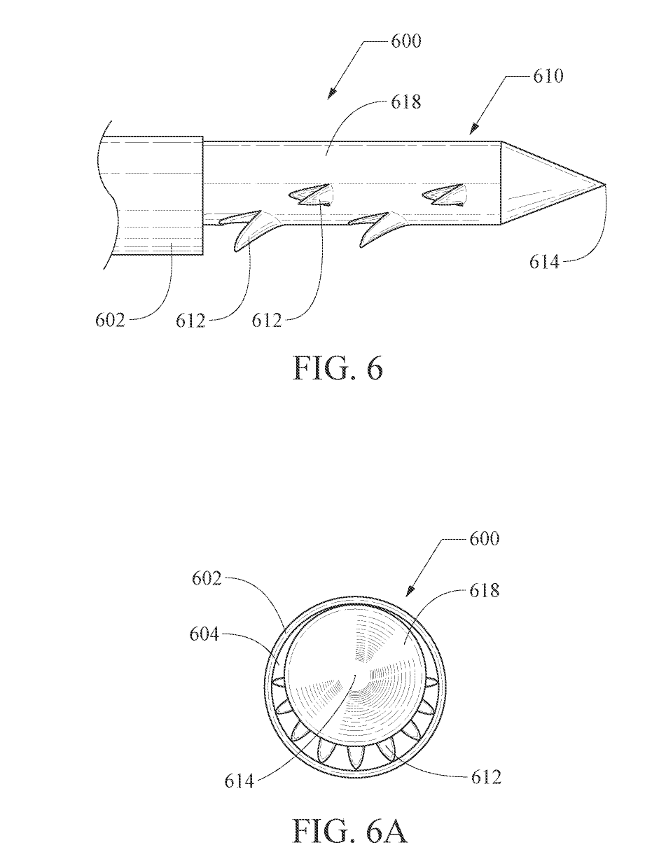

FIGS. 6 and 6A show an embodiment of a portion of a tissue sampling device 600. The device 600 includes an elongate member 602 disposed around a barbed member 610. The end of the elongate member 602 may be blunt and/or have a rounded end margin. The barbed member 610 includes differently-shaped barbs 612 that are disposed around one-half or less of the outer circumference of the body member 618. FIG. 6A shows a view taken from a distal end of the device 600, which more clearly shows the distal tip 614 of the barbed member 610, the position of the barbs around one-half or less of its outer circumference, and the disposition of the barbed member 610 in the lumen 604 of the elongate member 602. It will be appreciated that a barbed body member 618 may include a smaller outer diameter or a different geometry than is shown (e.g., non-cylindrical), and that such construction may allow for larger barbs around a portion of the outer surface of the body 618.

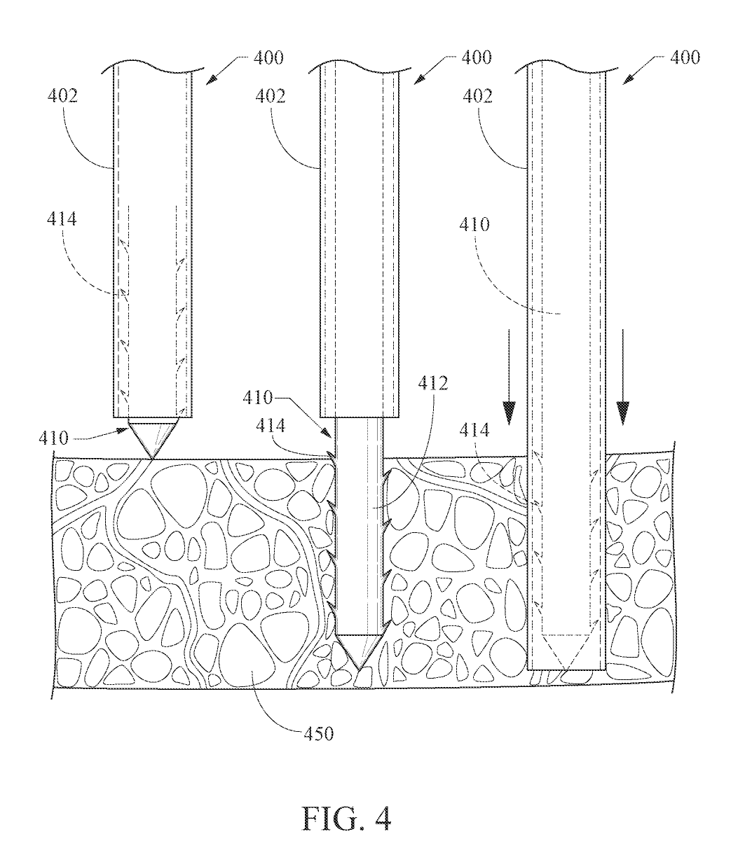

A method of use is described with reference to FIG. 4. Three different phases of the method are shown in FIG. 4. First (in the left portion of FIG. 4), a tissue-sampling device 400 is provided. The device includes an outer needle cannula 402 with a lumen. A barbed member 410 is slidably disposed through the lumen such that it may be slidably extended outward, then retracted back in. The barbed member 410 includes a central body 412 that has barbs 414 extending from it. The barbs 414 are formed wholly outside the outer circumference/outer diameter of the barbed body member 412. This is one other possible barbed member embodiment, but other barbed member embodiments, including the others described herein, may be used in the method without exceeding the scope of the claimed invention. In this tissue-sampling device and the other tissue-sampling devices disclosed and/or enabled herein, the barbed surface preferably provides an echogenic surface that will reflect ultrasound in a manner sufficiently distinguishable from tissue, fluids, and other surrounding materials that the tissue-sampling device and its overlying needle (and needle sheath, if present) may be visualized and navigated under ultrasound. However, the surface may also be dimpled or provided with another surface texturing or other regular or irregular surface feature(s) known in the art to provide enhanced echogenicity. It is preferable that the barbed region includes an echogenic region associated therewith, by which it is meant that the structure of the barbs and barb-adjacent surfaces provides desirable echogenicity and/or one or more structural features (e.g., surface dimples, embedded bubbles/vacant spaces/polymer beads, or other structural features) known to provide desirable echogenicity for ultrasound imaging is included.

The device 400 has been navigated (preferably under ultrasound rather than fluoroscopy) to a location adjacent a target tissue site 450 in a patient body. Next, as shown in the middle portion of FIG. 4, the barbed member 410 has been advanced into the target tissue 450 such that the barbs 414 engage the tissue 450. Then, the portion of tissue 450 engaged by the barbs 414 (together with adjacent tissue) may be captured. The tissue capture may be accomplished in one or both of the following ways: (1) As shown in the right portion of FIG. 4, the needle cannula 402 may be advanced over the barbed region, then the entire device 400 withdrawn with the captured tissue sample; and/or (2) The barbed member 410 may be retracted back into the position shown in the left portion of FIG. 4, bringing with it tissue captured in the barbs (not shown). The process may be repeated in a different location, to a different depth, or without even moving the needle as, particularly in the case of soft tissues, the tissues surrounding the just-sampled area will often “collapse” in and provide more tissue that can be engaged by the barbs 414.

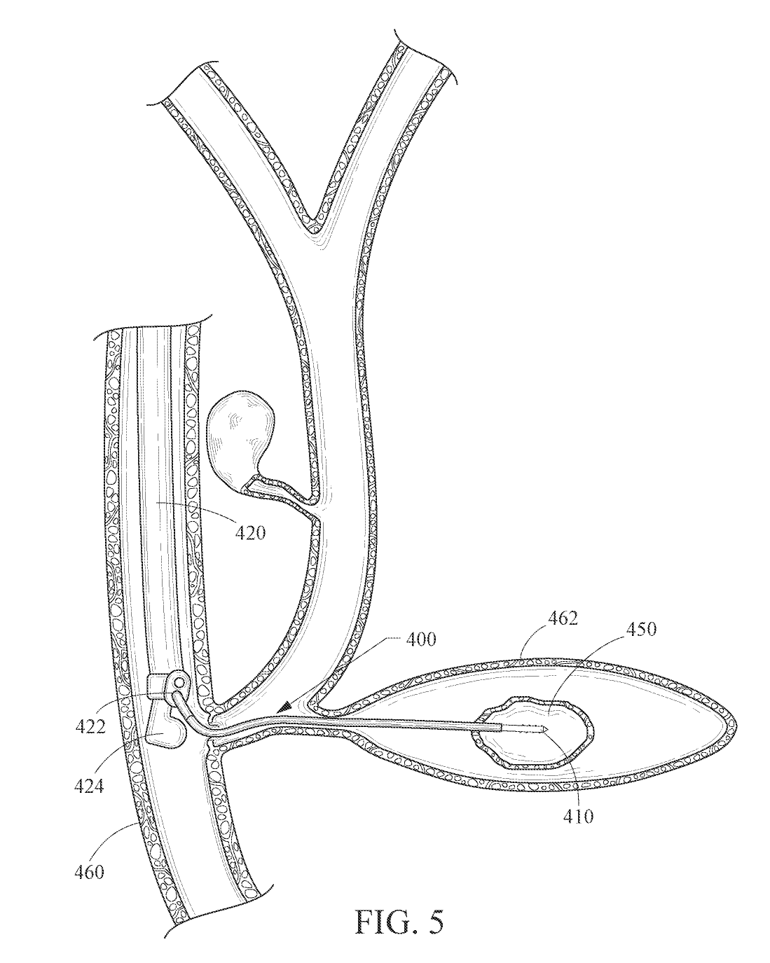

A further embodiment of the method just described with reference to FIG. 4 is described here with reference to FIGS. 4 and 5. In this further embodiment, an endoscope 420 is provided. The endoscope includes a working channel 422 through which the device 400 has been directed. The device 400 is shown as also including a needle sheath 404 from which the needle cannula 402 extends distally. The endoscope 420 is shown as having been directed into a patient's duodenum 460, and the device 400 is shown as having been directed to a tumor mass 450 in the pancreas 462. In a preferred embodiment, an ultrasound unit 424 is provided on the endoscope 420 and used to navigate the device 400 to the target site. The barbed member 410 is shown extended into the target tissue 450 in the manner described above with reference to the middle portion of FIG. 4. The tissue capture can be executed in the manner described above. For example, the needle cannula 402 may be advanced over the barbed member 410 (e.g., to incise tissue in a coring manner), then both withdrawn substantially simultaneously.

Those of skill in the art will appreciate that embodiments not expressly illustrated herein may be practiced within the scope of the claimed invention, including that features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention.

1. A tissue sampling device, comprising:
an outer elongate member comprising a longitudinal member lumen extending through a length thereof, and
a barbed member slidably disposed through the member lumen, the barbed member including
a central body member having a distal end and a central longitudinal body member axis, and
a plurality of barbs projecting outwardly from the central body member and forming a barbed region near the distal end where at least one of the barbs is angled away from the distal end;
where the barbed member is configured to be extended out of the distal end of the member lumen to engage the barbed region with a target tissue to be sampled, then retracted back thereinto with a tissue sample captured by at least one of the barbs; and
where the barbed member comprises an echogenic surface.

2. The tissue sampling device of claim 1, where the barbs define an overall outer diameter that is greater than an outer diameter defined by the central body member.

3. The tissue sampling device of claim 1, further comprising an elongate flexible needle shaft body extending proximally from the outer elongate member and being configured to pass through a working channel of an endoscope.

4. The tissue sampling device of claim 2, further comprising a needle sheath including a needle sheath lumen through which the needle shaft body is disposed.

5. The tissue sampling device of claim 2, further comprising an elongate flexible stylet body extending proximally from the body member.

6. The tissue sampling device of claim 4, further comprising an endoscope including a longitudinal working channel disposed therethrough, where the tissue sampling device extends through a length of the longitudinal working channel.

7. The tissue sampling device of claim 1, where at least one of the barbs is coarse such that it extends radially out from the body member in a manner where a radius from the longitudinal body axis to an outermost barb tip is at least the same as or greater than a distance equal to the body member outer diameter.

8. The tissue sampling device of claim 1, where at least one of the barbs includes a rounded tip.

9. The tissue sampling device of claim 1, where the barbed member comprises a beveled piercing distal tip.

10. The tissue sampling device of claim 1, where the plurality of barbs is disposed around one-half or less of an outer circumference of the body member.

11. The tissue sampling device of claim 1, where the outer elongate member comprises a cutting edge forming at least a portion of its distal tip.

12. The tissue sampling device of claim 11, where the outer elongate member is configured to incise tissue in a coring manner.

13. The tissue sampling device of claim 1, where the body member comprises portion having a generally non-cylindrical geometry.

14. The tissue sampling device of claim 13, where the body comprises a semi-cylindrical portion including a generally flat planar face and at least one of the plurality of barbs extends from the face.

15. The tissue sampling device of claim 1, where the barbs are formed wholly external of the body member outer diameter.

16. A method of obtaining a tissue sample, the method comprising the steps of:
providing the tissue sampling device of claim 1;
advancing a distal tip of the outer elongate member to a location adjacent a target tissue site in a patient body, navigating by ultrasound visualization of the echogenic region of the barbed member;
advancing the barbed region of the barbed member into the target tissue site; and
capturing tissue by advancing the needle over the barbed region, retracting the barbed region into the member lumen, or a combination thereof.

17. The method of claim 16, further comprising the step of repeating the steps of advancing into target tissue site and capturing tissue.

18. The method of claim 16, further comprising steps of
providing an endoscope with a working channel;
directing the endoscope such that the working channel provides access near the target tissue site;
directing the tissue sampling device distally through the working channel; and
where the step of advancing the distal tip of the needle to a location adjacent a target tissue site includes advancing a portion of the outer elongate member through and out of the working channel.

19. The method of claim 16, where the step of capturing tissue comprises advancing the distal tip of the needle over the barbed region, and further comprises withdrawing the needle and barbed member from the target tissue site substantially simultaneously.

20. A tissue sampling system, comprising:
an elongate flexible needle sheath including a needle sheath lumen disposed longitudinally therethrough;
a needle member disposed slidably through the needle sheath and comprising
an elongate flexible needle shaft body configured to pass through a working channel of an endoscope and
an outer needle cannula disposed at a distal end of the needle shaft body and housing a longitudinal needle lumen though its length, and
a sampling member disposed slidably through the needle lumen and comprising
an elongate flexible stylet body and
a barbed portion extending distally from the stylet body and including a central body with a distal end, a central longitudinal body axis, and a plurality of barbs forming a barbed region where at least one of the barbs is angled away from the distal end;
where the barbed region is configured to be slidably extended out of the distal end of the needle lumen, then retracted back thereinto; and
where the barbed region comprises an echogenic surface associated with the barbs and barb-adjacent surfaces of the central body.
